(19) United States
(12) Patent Application Publication
(10) Pub. No.: US 2024/0053342 A1
(43) Pub. Date: Feb. 15, 2024

(54) ANTITHROMBIN HEPARAN SULFATE FOR DETECTION AND TREATMENT OF CANCER

(71) Applicant: The Regents of the University of California, Oakland, CA (US)

(72) Inventors: Ryan Weiss, Athens, GA (US); Thomas Mandel Clausen, San Diego, CA (US); Jeffrey D. Esko, San Diego, CA (US)

(21) Appl. No.: 18/257,838

(22) PCT Filed: Dec. 20, 2021
(86) PCT No.: PCT/US2021/064282

§ 371 Date: Jun. 15, 2023

Related U.S. Application Data
(60) Provisional application No. 63/127,194, filed on Dec. 18, 2020.
(60) Provisional application No. 63/127,198, filed on Dec. 18, 2020.

(51) Int. Cl. G01N 33/574; A61K 45/06; A61K 38/48; A61P 35/00

(52) U.S. Cl. CPC G01N 33/57484; A61K 45/06; A61K 38/4833; A61P 35/00; G01N 2474/20; G01N 2333/8128

(57) ABSTRACT

Methods of detecting and treating an epithelial carcinoma, such as pancreatic ductal adenocarcinoma cancer or bladder cancer, expressing antithrombin-binding heparan sulfate (HS^{AT}) in a subject, comprising combining a biological sample of the subject containing HS^{AT} with antithrombin and detecting binding of HS^{AT} and the antithrombin, optionally determining that the antithrombin inhibits factor Xa.

### CROSS-REFERENCE TO RELATED APPLICATIONS

The present application claims priority to U.S. Provisional Application No. 63/127,194 and U.S. Provisional Application No. 63/127,198, both filed on Dec. 18, 2020, the contents of which are hereby incorporated by reference herein.

### GOVERNMENT SPONSORSHIP

This invention was made with government support under grant No. CA199292 awarded by the National Institutes of Health. The government has certain rights in the invention.

### TECHNICAL FIELD

The present invention relates to heparan sulfate cancer diagnostics and treatment.

### BACKGROUND

Pancreatic ductal adenocarcinoma (PDAC) is one of the most fatal malignancies, with a ˜4% 5-year survival rate and very limited diagnostic and therapeutic strategies. PDAC tumors have a characteristic dense fibrotic extracellular matrix (ECM) with a highly elevated content of glycosaminoglycans (GAGs), such as heparan sulfate (HS) (Oberstein et al, 2013). Early diagnosis improves the chances for successful therapy, but by the time PDAC is discovered the tumors have become invasive and/or metastatic.

Early bladder cancer diagnosis improves the chances for successful therapy. Current methods for detection of bladder cancer include urinalysis, cystoscopy, biopsy, and imaging (e.g., computed tomography, Intravenous pyelogram, retrograde pyelogram, magnetic resonance imaging, x-ray, and ultrasound) are either insufficient or very invasive.

Antithrombin is a potent inhibitor of thrombin and factor Xa in the coagulation cascade. It is known that heparin acts as an anticoagulant by binding to antithrombin, causing a conformational change, and thereby significantly enhancing antithrombin's ability to inhibit factor Xa and thrombin in the coagulation cascade (Teien et al 1976; Shworak et al 2010; Jin et al 1997). Heparin is widely used as an anticoagulant in human patients during surgery and in outpatient care (Glass et al 2018). Heparan sulfate is related in structure to heparin and binds to antithrombin dependent on 3-O-sulfation of a glucosamine residue within a pentasaccharide binding motif within the HS polysaccharide chains (Ersdal-Badju et al. 1997). This unique epitope is referred to as HS^{AT}.

### SUMMARY OF THE INVENTION

The invention leverages antithrombin binding to HS^{AT} on the cell surface or in the secreted fraction of epithelial carcinomas, such as human PDAC and bladder cancer, for diagnosis, prognosis and treatment of a subject in need. Antithrombin-binding heparan sulfate (HS^{AT}) can be detected in biological samples from the subject including urine, blood or feces. Antithrombin can be used as a HS^{AT} specific stain in immunohistochemistry or as a tracer for in vivo imaging of HS^{AT}-positive tumors. Antithrombin can also be conjugated with other diagnostic or therapeutic agents, such as cytotoxic compounds, for targeted delivery to the cancerous cells in therapy.

Pancreatic ductal adenocarcinoma (PDAC), and other carcinoma cancer cells (bladder, prostate), produce a distinct type of antithrombin-binding heparan sulfate (HS^{AT}) that can be targeted by antithrombin. The Hs3st1 gene encodes HS 3-O-sulfotransferase-1, whose rate limiting action regulates cellular production of HS^{AT}. This invention provides utilizing HS^{AT}, antithrombin, and the anti-Factor Xa assay, for cancer diagnostic and therapeutic purposes. The invention provides for specific therapeutic targeting of HS^{AT} containing tissues with antithrombin, as a method of cancer treatment.

Heparan sulfate is expressed on the cell surface, in the extracellular matrix, and is secreted into the fluid compartments. Specific expression of HS^{AT}, targetable by antithrombin, can be detected in tissue biopsies, as well as in non-invasive liquid biopsies such as plasma, urine, or fecal juice. The detection of HS^{AT} can be performed using the clinically validated anti-Factor Xa assay for that is used to measured heparin in patients receiving heparin anticoagulant therapy. The invention provides that HS^{AT} is a marker for early grades of pancreatic and bladder cancer, but also other types of cancer as well, such as prostate cancer, and other epithelial carcinomas. The invention provides that the level of HS^{AT} detected can be correlated with the stage or severity of the cancer, and hence can indicate modifications to known and future therapeutic cancer treatments. The invention provides that HS^{AT} can be used as a marker for diagnostics and that the specific binding of antithrombin can be utilized for the therapeutic targeting of HS^{AT} positive tumors. The invention also provides methods for production and purification of HS^{AT} in cultured mammalian cells, as a source of material for clinical anticoagulation.

### BRIEF DESCRIPTION OF THE DRAWINGS

FIGS. 1a-1d show HS3ST1 expression in tumors in patient cohorts. FIG. 1a shows HS3ST1 expression in PDAC and pancreas of healthy patients, data derived from the TCGA. Expression is shown across clinical stages. The data clearly shows an increase in HS3ST1 expression in pancreatic cancer compared to normal, with an increase in early clinical stages. The TCGA set contains only few healthy pancreas samples, so FIG. 1b shows comparison of HS3ST1 expression in PDAC taken from TCGA, containing 223 samples from PDAC patients, compared to expression in healthy pancreas as extracted from GTEX, containing expression data from 328 healthy pancreatic specimens. This clearly shows an increase in HS3ST1 expression in PDAC. Expression is shown as the density of log 2 transformed TPM values, from the two datasets. FIG. 1c shows that HS3ST1 expression is linked to poor outcome in PDAC, measured as relapse-free survival in a patient cohort, from TCGA. FIG. 1d shows analysis of HS3ST1 expression in single cell RNASeq dataset from human PDAC, published by Peng et al. The data shows that HS3ST1 is specifically upregulated in the malignant cells, with a preference for expression in the PDAC PanIN precursor lesions.

FIG. 2 shows antithrombin (AT) binding to HS^{AT} on PDAC cells. CHO WT cells do not express HS3ST1 and is used as a negative control. CHO3.1 expresses HS3ST1 and represents the positive control. The samples include a murine PDAC derived from the KPC mouse; a patient derived primary PDAC; a human primary metastatic PDAC derived from the circulating tumor cell (CTC) fraction of a patient. FG and Capan-2 are ATCC lines. Heparin lyase (HSase) treatment is used to verify the HS-dependence of AT binding. This data verifies the presence of HS^{AT} on the PDAC cells.

FIG. 3 shows HS^{AT} in plasma from PDAC patients. Plasma from five PDAC patients and five non-neoplastic disease individuals were screened for HS^{AT} using the anti-FXa assay. Readout is given as equivalent unfractionated heparin (UFH). The data shows that HS^{AT} was found in the plasma from PDAC patients, but not in healthy plasma.

FIG. 4 shows anti-FXa assay detection of HS^{AT} in urine from bladder cancer patients. This data illustrates the presence of HS^{AT} in urine from bladder cancer patients.

FIG. 5 shows test of HS isolated from PDAC cells, in its ability to activate antithrombin in the anti-FXa assay. HS was isolated from KPC and KPC HS3ST1−/− cells and compared to heparin for the ability to activate antithrombin in the anti-FXa assay. This shows that the HS^{AT} in PDAC cells is capable of activating antithrombin and enables AT in its anticoagulative role, and that this activity is dependent on HS3ST1 expression.

FIG. 6 shows test of HS isolated from bladder cancer cells, in its ability to activate antithrombin in the anti-FXa assay. HS was isolated from bladder cancer cell lines and compared to heparin for the ability to activate antithrombin in the anti-FXa assay. This shows that the HS^{AT} in bladder cancer cells is capable of activating antithrombin and enables AT in its anticoagulative role.

FIGS. 7a-7c show HS^{AT} staining in formalin-fixed paraffin-embedded tissue using antithrombin. FIG. 7a, Primary tumor specimen from PDAC patient (40×). FIG. 7b, Serial tissue section treated with HSase to remove HS (40×). FIG. 7c, Acinar cell compartment in normal pancreas (20×); only blood vessels stain. Antithrombin staining is shown in red greyscales. Green greyscales represents autofluorescence. Blue greyscales is Hoechst nuclear stain. This shows a specific stain of PDAC cells in pancreatic cancer.

FIGS. 8a-8b show HS^{AT} staining of PDAC precursor lesions (PanINs). FIG. 8a, Example of a resected PanIN lesion, as part of a larger TMA. FIG. 8b, serial tissue section treated with HSase to remove HS. Staining was performed in FFPE tissue using AT detected by anti-AT and an HRP conjugated tertiary antibody. Development was performed with DAB. Mayer's counterstain for nuclei. This shows HS^{AT} in early cancer development.

FIGS. 9a-9c show targeting of HS^{AT} in vivo using fluorescently labelled AT. FIG. 9a, shows localization of I.V. injected AT in mouse models of spontaneous PDAC development (KPC mice) (Hingorani et. al. 2005). Organs from the mice were extracted and scanned for fluorescence. FIG. 9b, shows localization of I.V. injected AT in mice injected I.V. with murine PDAC cells to generate tumor colonies in the lungs. Organs from mice with lung metastases and a healthy mouse, and a fourth animal showing autofluorescence. FIG. 9c, shows localization of I.V. injected AT in mice carrying subcutaneous tumors of KPC cells. Genotype of the KPC cells is indicated to the right. Mice were injected with 10 μg AT-Alexa 750. Organs were scanned 24 hr post injection. Alexa-750 staining is shown in green greyscales. General tissue autofluorescence is shown in red greyscales.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including.” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone. A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human, mammalian or other animal subject to be diagnosed or treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

Heparan sulfate is a linear polysaccharide chain that is added post-translationally to proteins in almost every cell type. It comprises a 4-sugar primer sequence that is covalently linked at one end to the protein. The other end of the primer sequence is extended by the addition of repeating disaccharide units comprising N-acetyl glucosamine and glucuronic acid. Heparan sulfate chains can vary in length, from about 40 to about 300 sugar residues. The sugars that make up heparan sulfate are highly sulfated, and patterns of sulfation along the chain create negatively-charged regions that serve as binding sites for various proteins. When proteins are bound by heparan sulfate, their activity, bioavailability, and clearance may be altered. Heparin is similar to heparan sulfate; however, it is more highly sulfated and shorter in length.

Antithrombin-binding heparan sulfate (HS^{AT}) is a heparan sulfate that binds to antithrombin.

In embodiments, the invention provides methods of detecting a cancer in a subject expressing HS^{AT} comprising combining a biological sample from the subject containing HS^{AT} with antithrombin, and detecting binding of HS^{AT} to the antithrombin, thereby detecting the cancer in the subject.

In embodiments, the invention provides that the HS^{AT} is detected at a higher level than in a control subject. In embodiments, the invention provides that the HS^{AT} is detected by further determining that the antithrombin inhibits factor Xa.

In embodiments, the invention provides that the biological sample is a tissue biopsy, urine, blood or feces. In embodiments, the invention provides the HS^{AT} is detected by further detecting the antithrombin as a stain in immunohistochemistry, or as a tracer for in vivo imaging.

In embodiments, the invention provides that the cancer is pancreatic ductal adenocarcinoma (PDAC). In embodiments, the invention provides for earlier detection and/or treatment of PDAC or Pancreatic intraepithelial neoplasia (PanIN). In embodiments, the invention provides that the cancer is bladder cancer, prostate cancer or pancreatic cancer. In embodiments, the invention provides that the cancer is an epithelial carcinoma.

In embodiments, the invention provides further methods of treatment of cancer comprising administering to the subject in need thereof an effective amount of antithrombin conjugated to a therapeutic agent, such as a cytotoxic compound used in cancer therapy, for targeted delivery and binding to HS^{AT} in situ.

In embodiments, the invention provides a method of detecting antithrombin-binding heparan sulfate (HS^{AT}) in a subject comprising combining a biological sample of the subject containing HS^{AT} with antithrombin and detecting binding of HS^{AT} and the antithrombin, thereby detecting HS^{AT} in the subject.

In embodiments, the invention provides a method of treating a cancer expressing antithrombin-binding heparan sulfate (HS^{AT}) in a subject comprising detecting HS^{AT} binding to antithrombin in a biological sample of the subject, and administering an effective amount of a cancer treatment to the subject. In embodiments, the cancer treatment is an effective amount of antithrombin conjugated to a cancer therapy agent, such as a cytotoxic chemotherapeutic agent. In embodiments, the increased level of HS^{AT} binding to antithrombin informs the increased level of administration of the effective amount of the cancer treatment.

In embodiments, the invention provides methods of producing anticoagulant antithrombin-binding heparan sulfate (HS^{AT}), comprising culturing mammalian bladder cancer cells and purifying HS^{AT} from the cultured cells. In embodiments, the invention provides that the HS^{AT} is isolated from the surface of the cells. In embodiments, the invention provides that the anticoagulant heparan sulfate is isolated from a secreted fraction of the cells. In embodiments, the invention provides that the cells are RT4, RT112, or UC-9 or PDAC-354, murine CHX1990, circulating tumor cell derived C76, FG and Capan-2 as well as other bladder and PDAC cell lines. In embodiments, the invention provides that the HS^{AT} has anticoagulant activity dependent on antithrombin.

### EXAMPLES

### Example 1: Pancreatic Cancer

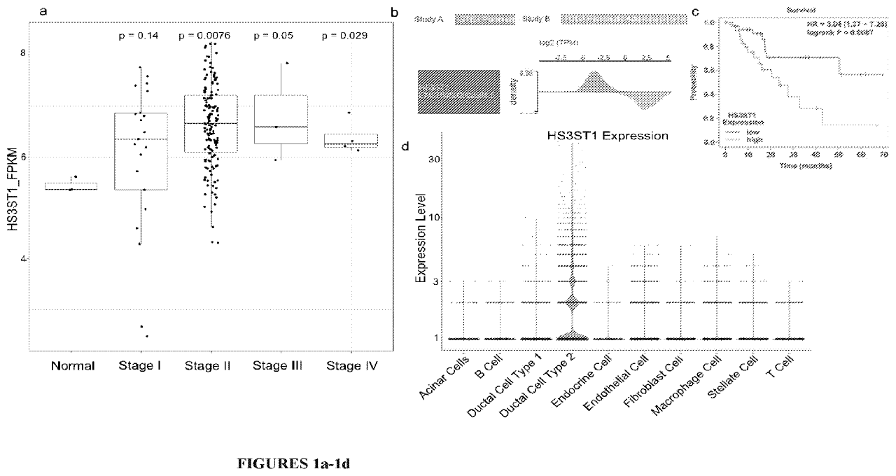

It has been discovered that HS3ST1, the enzyme responsible for HS^{AT} production, is overexpressed in pancreatic cancer, suggesting the presence of HS^{AT} in the tumor site. FIGS. 1a-1d show HS3ST1 expression in tumors in patient cohorts. FIG. 1a shows HS3ST1 expression in PDAC and pancreas of healthy patients, data derived from the TCGA. Expression is shown across clinical stages. The data clearly shows an increase in HS3ST1 expression in pancreatic cancer compared to normal, with an increase in early clinical stages. The TCGA set contains only few healthy pancreas samples so FIG. 1b shows comparison of HS3ST1 expression in PDAC taken from TCGA, containing 223 samples from PDAC patients, compared to expression in healthy pancreas as extracted from GTEX, containing expression data from 328 healthy pancreatic specimens. This clearly shows an increase in HS3ST1 expression in PDAC. Expression is shown as the density of log 2 transformed TPM values, from the two datasets. FIG. 1c shows that HS3ST1 expression is linked to poor outcome in PDAC, measured as relapse-free survival in a patient cohort, from TCGA. FIG. 1d shows analysis of HS3ST1 expression in single cell RNASeq dataset from human PDAC, published by Peng et al. 2019. The data shows that HS3ST1 is specifically upregulated in the malignant cells, with a preference for expression in the PDAC PanIN precursor lesions.

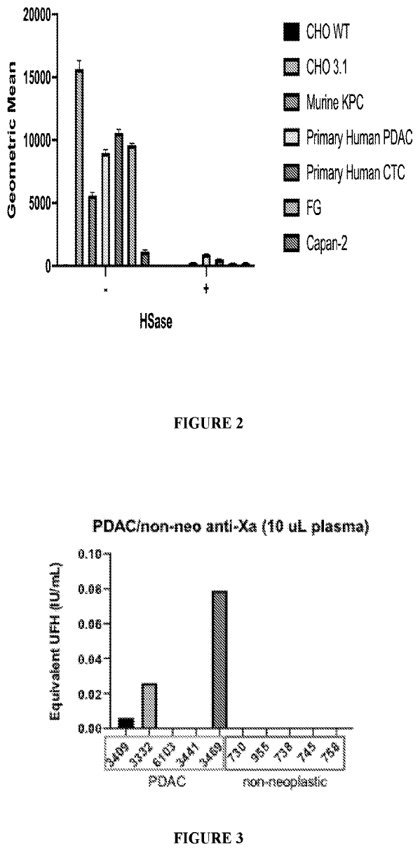

To investigate whether pancreatic cancer cells do indeed produce and present HS^{AT} at their surface, HS^{AT} were stained using antithrombin on a number of PDAC derived human and murine cells lines; FG and Capan-2 and a number of primary human and murine tumor derived lines, including human PDAC-354, murine CHX1990, and circulating tumor cell derived C76 (see FIG. 2).

Heparan sulfate is present on cell surfaces and is secreted into the extracellular matrix. Thus, if HS^{AT} is expressed in tumors it is likely that some HS^{AT} will be present in plasma or other liquid biopsies. To investigate if HS^{AT} is present in plasma from pancreatic cancer patients, plasma from five PDAC patients and five non-neoplastic disease individuals was screened using the anti-FXa assay, for HS^{AT} detection. Readout is given as equivalent unfractionated heparin (UFH). The data shows that HS^{AT} was found in the plasma from PDAC patients, but not in healthy plasma (FIG. 3).

The presence of HS^{AT} in heparin gives it the ability to activate antithrombin that can then inactivate thrombin and FXa in the coagulation cascade. To test if HS^{AT} produced by PDAC cells can activate antithrombin, HS from PDAC cells was isolated and tested for an ability to activate antithrombin in the anti-FXa assay. HS was isolated from KPC and KPC HS3ST1−/− cells and compared to heparin for the ability to activate antithrombin in the anti-FXa assay. This shows that the HS^{AT} in PDAC cells is capable of activating antithrombin and enables AT in its anticoagulative role, and that this activity is dependent on HS3ST1 expression (FIG. 5).

To investigate whether HS^{AT} is present in PDAC tissue, a method was developed to identify HS^{AT} in tissue sections by incubation of tissue sections with purified human AT, followed by reaction with a murine anti-human AT antibody and a secondary goat anti-mouse IgG tagged with Alexa594. Staining of primary PDAC patient tumor biopsies revealed that HS^{AT} is present on the basolateral side and basement membrane of the polarized tumor cells making up the tumor nodules in differentiated PDAC samples (FIG. 7a, red staining). The staining is specific for HS^{AT} because binding was prevented by treatment with heparin lyases (HSase) that remove HS (FIG. 7b). Acinar cells in the normal pancreas do not bind AT and only weak staining on the luminal side of the microvasculature is observed, consistent with prior studies showing that cultured endothelial cells express low levels of AT-binding heparan sulfate (FIG. 7c). This shows that HSAT is expressed by PDAC and only to a limited extend by normal pancreatic tissue.

To investigate HS^{AT} across different stages of PDAC development, slides of specifically dissected PDAC precurser PanIN lesions were obtained. This revealed a marked expression of HS^{AT} in the validated PanIN lesions, suggesting that HS^{AT} may be an early marker of PDAC development (FIG. 8a,b). Staining was performed in FFPE tissue using AT detected by anti-AT and an HRP conjugated tertiary antibody. Development was performed with DAB. Mayer's counterstain for nuclei. This shows HS^{AT} in early cancer development.

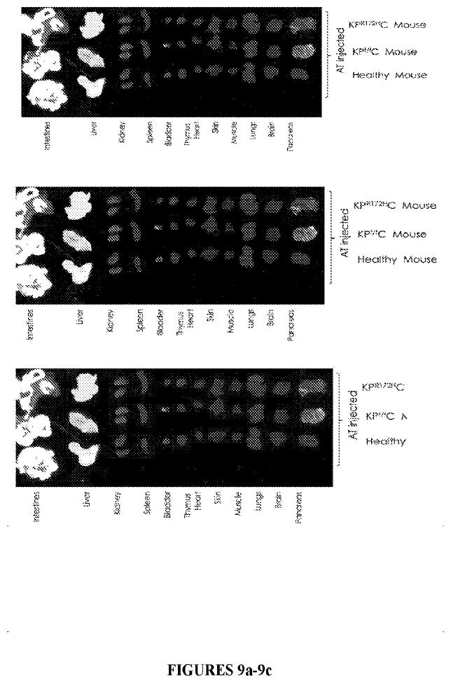

To test whether antithrombin can be used as an anti-HS^{AT} targeting reagent in cancer therapy and diagnostics. We performed in vivo experiments in which KPC (mice that develop spontaneous PDAC) mice with palpable tumors were injected with AT tagged with Alexa-750. After 24 hours organs were removed and scanned using a fluorescence reader. Remarkable staining of the tumor was observed with little signal seen in other organs, except the liver and kidneys (FIG. 9a). Most importantly no localization was seen in the pancreas of a healthy mouse. Similar tumor specific localization was seen in an experimental model for metastasis, in which murine KPC cells are injected through the tail vein to colonize the lung (FIG. 9b). Finally, the homing was shown to be HS^{AT} specific as knockout of Hs3st1 or Ext1, which is required for general HS synthesis, significantly reduced tumor localization (FIG. 9c).

### Example 2—Bladder Cancer

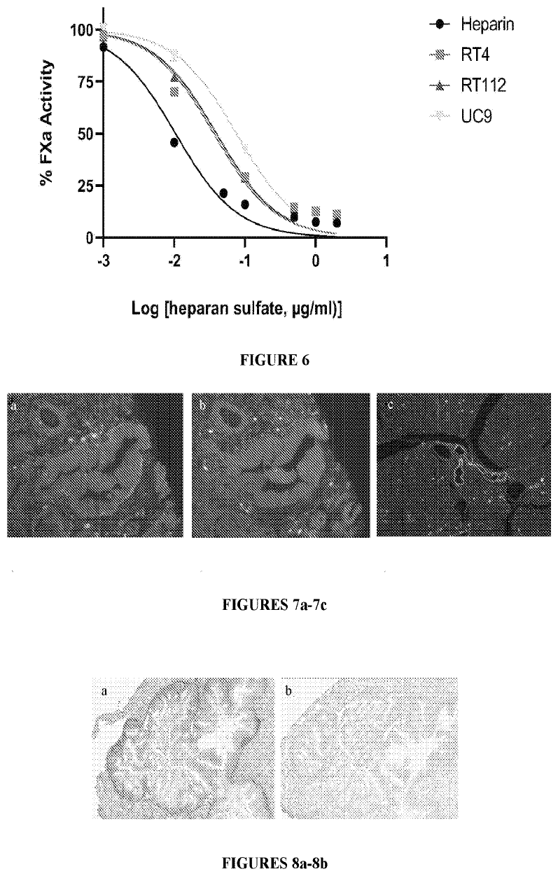

Heparin isolated from pig mucosa or bovine lung tissue is currently used as an anticoagulant in human patients during surgery and in outpatient care. It is known that heparin acts as an anticoagulant by binding to the serum protein antithrombin, causing a conformational change, and thereby significantly enhancing antithrombin's ability to inhibit factor Xa in the coagulation cascade. Additionally, it is known that heparan sulfate can also bind to antithrombin in vivo and this is dependent on 3-0 sulfation of HS. Heparan sulfate can also bind to antithrombin in vivo and this is dependent on 3-O-sulfation within a pentasaccharide binding motif within the HS polysaccharide chains. It was discovered that heparan sulfate isolated from the cell surface or secreted fraction of cultured human bladder cancer cells (RT4, RT112, UC-9) has anticoagulant activity dependent on antithrombin. FIG. 6 shows test of HS isolated from bladder cancer cells, in its ability to activate antithrombin in the anti-FXa assay. HS was isolated from bladder cancer cell lines and compared to heparin for the ability to activate antithrombin in the anti-FXa assay. This shows that the HS^{AT} in bladder cancer cells cells is capable of activating antithrombin and enables AT in its anticoagulative role.

Initial experiments focused on isolating heparan sulfate from cultured human bladder cancer cell lines (RT4, RT112, and UC-9) and utilizing LC-MS methods to quantify and characterize the material for anti-FXa activity. It was observed that certain human bladder cell lines uniquely produce heparan sulfate with anticoagulant properties similar to pharmaceutical-grade heparin.

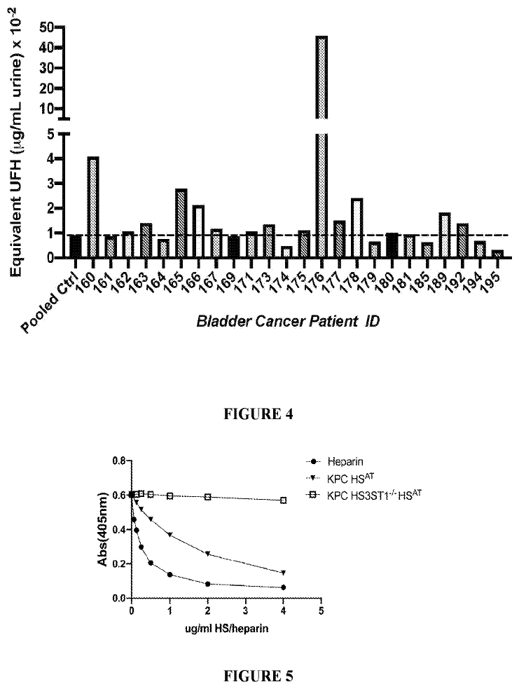

Bladder cancers are uniquely situated at the bladder wall in close proximity to the urine filled lumen. For this reason, it is possible that HS^{AT} is secreted into the urine and can be used as a biomarker. FIG. 4 shows anti-FXa assay detection of HS^{AT} in urine from bladder cancer patients. This data illustrates the presence of HS^{AT} in urine from bladder cancer patients.

Additionally, it is believed cancer cells in patients with interstitial carcinoma of the bladder might secrete anticoagulant heparan sulfate into the urine, which could then be used as a biomarker for early detection of bladder cancer in human patients via a simple clinical laboratory test for anti-FXa activity. To test this, urine from a cohort of bladder cancer patients was screened for the presence of HS^{AT} using the anti-FXa assay (FIG. 4).

### REFERENCES

Peng J, Sun B F, Chen C Y, et al. Single-cell RNA-seq highlights intra-tumoral heterogeneity and malignant progression in pancreatic ductal adenocarcinoma [published correction appears in Cell Res. 2019 Aug. 13;:]. Cell Res. 2019; 29(9):725-738. doi:10.1038/s41422-019-0195-y.
Hingorani S R, Wang L, Multani A S, Combs C, Deramaudt T B, Hruban R H, Rustgi A K, Chang S, Tuveson D A. Trp53R172H and KrasG12D cooperate to promote chromosomal instability and widely metastatic pancreatic ductal adenocarcinoma in mice. Cancer Cell. 2005 May; 7(5):469-83. doi: 10.1016/j.ccr.2005.04.023. PMID: 15894267.
Oberstein P E, Olive K P. Pancreatic cancer: why is it so hard to treat? Therap Adv Gastroenterol. 2013 Jul; 6(4):321-37. doi: 10.1177/1756283X13478680. PMID: 23814611; PMCID: PMC3667471.
Arne N. Teien, Ulrich Abildgaard, Magnus Höök, The anticoagulant effect of heparan sulfate and dermatan sulfate, Thrombosis Research, Volume 8, Issue 6, 1976, Pages 859-867.
Shworak N W, Kobayashi T, de Agostini A, Smits N C. Anticoagulant heparan sulfate to not clot—or not? Prog Mol Biol Transl Sci. 2010; 93:153-78. doi: 10.1016/S1877-1173(10)93008-1. PMID: 20807645.
Jin, L., Abrahams, J. P., Skinner, R., Petitou, M., Pike, R. N., and Carrell, R. W. (1997) The anticoagulant activation of antithrombin by heparin. Proc Natl Acad Sci USA 94, 14683-14688
Glass, C. A. (2018). Recombinant Heparin-New Opportunities. Frontiers in Medicine, 5(341). doi:10.3389/fmed.2018.00341
Ersdal-Badju E, Lu A, Zuo Y, Picard V, Bock SC. Identification of the antithrombin III heparin binding site. J Biol Chem. 1997 Aug. 1; 272(31):19393-400. doi: 10.1074/jbc.272.31.19393. PMID: 9235938.

What is claimed is:

1. A method of detecting antithrombin-binding heparan sulfate (HS^{AT}) in a biological sample comprising combining the biological sample with antithrombin and detecting binding of HS^{AT} and the antithrombin, thereby detecting HS^{AT} in the sample.

2. The method of claim 1, wherein the HS^{AT} is detected by determining that the antithrombin/HS^{AT} complex inhibits factor Xa.

3. The method of claim 1, wherein the HS^{AT} is detected by further detecting the antithrombin as a stain in immunohistochemistry, or as a tracer in in vivo imaging.

4. The method of claim 1, wherein biological sample is a liquid biopsy, urine, blood or feces.

5. The method of claim 4, wherein the biological sample is from a subject with pancreatic ductal adenocarcinoma (PDAC), bladder cancer or prostate cancer.

6. A method of detecting a cancer expressing antithrombin-binding heparan sulfate (HS^{AT}) in a subject comprising combining a biological sample of the subject with antithrombin, and detecting binding of HS^{AT} and the antithrombin, thereby detecting the cancer in the subject.

7. The method of claim 6, wherein the HS^{AT} is detected at a higher level than in a control subject.

8. The method of claim 6, wherein the HS^{AT} is detected by determining that the antithrombin inhibits factor Xa.

9. The method of claim 6, wherein biological sample is a biopsy, urine, blood or feces.

10. The method of claim 6, wherein the HS^{AT} is detected by further detecting the antithrombin as a stain in immunohistochemistry, or as a tracer in in vivo imaging.

11. The method of claim 6, wherein the cancer is pancreatic ductal adenocarcinoma (PDAC), bladder cancer or prostate cancer.

12. A method of treating a cancer expressing antithrombin-binding heparan sulfate (HS^{AT}) in a subject comprising combining a biological sample of the subject containing HS^{AT} with antithrombin, detecting binding of HS^{AT} and the antithrombin, and treating the subject for the cancer by administering an effective amount of cancer therapy.

13. The method of claim 12, wherein the HS^{AT} is detected at a higher level than in a control subject.

14. The method of claim 12, wherein the HS^{AT} is detected by further determining that the antithrombin inhibits factor Xa.

15. The method of claim 12, wherein biological sample is a biopsy, urine, blood or feces.

16. The method of claim 12, wherein the HS^{AT} is detected by further detecting the antithrombin as a stain in immunohistochemistry, or as a tracer for in vivo imaging.

17. The method of claim 12, wherein the cancer therapy comprises an effective amount of thrombin conjugated to a cancer therapy agent.

18. A method of producing antithrombin-binding heparan sulfate (HS^{A}T), comprising culturing mammalian bladder cancer cells and purifying HS^{AT} from the cultured cells.

19. The method of claim 18, wherein the HS^{AT} is isolated from the surface of the cells.

20. The method of claim 18, wherein the HS^{AT} is isolated from a secreted fraction of the cells.
